(19) United States
(12) Patent Application Publication
(10) Pub. No.: US 2009/0171094 A1
(43) Pub. Date: Jul. 2, 2009

(54) 1-BENZYL-4-[(5, 6-DIMETHOXY- 1- INDANON)- 2- YL]-METHYL PIPERIDINE P-TOLUENESULFONATE OR CRYSTAL THEREOF

(76) Inventors: Kazuhide Ashizawa, Tsukuba (JP); Akio Imai, Kamisu (JP)

Correspondence Address: BIRCH STEWART KOLASCH & BIRCH, PO BOX 747, FALLS CHURCH, VA, 22040-0747, US

(21) Appl. No.: 11/994,494

(22) PCT Filed: Jul. 12, 2006
(86) PCT No.: PCT/JP2006/314212

§ 371 Date: Jan. 2, 2008

Related U.S. Application Data
(60) Provisional application No. 60/700,732, filed on Jul. 20, 2005.

(30) Foreign Application Priority Data
Jul. 15, 2005 (JP) 2005-206388

(51) Int. Cl. C07D 211/32

(52) U.S. Cl. 546/206

(57) ABSTRACT

1-Benzyl-4-[(5,6-dimethoxy-1-indanon)-2-yl]methylpiperidine p-toluenesulfonate or a solvate thereof.

### TECHNICAL FIELD

The present invention relates to donepezil p-toluenesulfonate or a crystal thereof having acetylcholinesterase inhibitory effect which is useful as a preventive or therapeutic agent for various types of senile dementia, etc.

### BACKGROUND ART

Donepezil hydrochloride (chemical name: 1-benzyl-4-[(5,6-dimethoxy-1-indanon)-2-yl]methylpiperidine hydrochloride) is a preventive or therapeutic agent for various types of senile dementia having acetylcholinesterase inhibitory effect and particularly it is extremely useful as a preventive or therapeutic agent for Alzheimer's type senile dementia (see Patent Document 1).

As inorganic salts of donepezil, 1-benzyl-4-[(5,6-dimethoxy-1-indanon)-2-yl]methylpiperidine hydrochloride and a crystal thereof (Patent Document 2) and 1-benzyl-4-[(5,6-dimethoxy-1-indanon)-2-yl]methylpiperidine hydrobromide and a crystal thereof (Patent Document 3) are known. Also, as organic salts of donepezil, organic acid salts including p-toluensulfonate (Patent Documents 4 to 7) were described after the filing of patent applications relating to the claim for priority of this patent application.

### [Patent Document 1] JP-A-64-79151

### [Patent Document 2] WO97/46527A

### [Patent Document 3] WO2004/099142A

### [Patent Document 4] WO2006/030249A

### [Patent Document 5] WO2006/032432A

### [Patent Document 6] WO2006/001031A

### [Patent Document 7] WO2006/035433A

### DISCLOSURE OF INVENTION

A salt and a crystal to be used as pharmaceutical raw materials are required to have properties easy to handle in industrial production.

As a result of having performed studies intensively, the present inventors have found the following new salt and completed the present invention. That is, the present invention relates to donepezil p-toluenesulfonate or a solvate thereof, or a crystal thereof.

Namely, the present invention includes the followings.

(1) 1-Benzyl-4-[(5,6-dimethoxy-1-indanon)-2-yl]methylpiperidine p-toluenesulfonate or a solvate thereof;
(2) A crystal of 1-benzyl-4-[(5,6-dimethoxy-1-indanon)-2-yl]methylpiperidine p-toluenesulfonate or a solvate thereof having diffraction peaks at diffraction angles (2θ±0.2°) of 15.7° and 22.2° in powder X-ray diffraction;
(3) The crystal according to the above (2) wherein the crystal has further diffraction peaks at diffraction angles (2θ±0.2°) of 7.8° and 14.3°;
(4) A process for preparing the crystal of the above (2) characterized in that crystallization is performed using one or two solvents selected from the group consisting of alcohols, ethers and water;
(5) A drug comprising the salt or solvate thereof or crystal thereof according to any one of the above (1) to (3);
(6) A preventive or therapeutic agent for a disease to which acetylcholinesterase inhibitory effect is effective, wherein the agent comprises as an active ingredient the salt or solvate thereof or crystal thereof according to any one of the above (1) to (3);
(7) A preventive or therapeutic agent for senile dementia, wherein the agent comprises as an active ingredient the salt or solvate thereof or crystal thereof according to any one of the above (1) to (3);
(8) A preventive or therapeutic agent for Alzheimer's disease, wherein the agent comprises as an active ingredient the salt or solvate thereof or crystal thereof according to any one of the above (1) to (3);
(9) A pharmaceutical composition comprising the salt or solvate thereof or crystal thereof according to any one of the above (1) to (3).

### BRIEF DESCRIPTION OF DRAWINGS

FIG. 1 is a drawing showing a powder X-ray diffraction pattern of the crystal obtained in Example 1.

### BEST MODE FOR CARRYING OUT THE INVENTION

Hereinafter, we explain the meanings of terms, signs, etc. used in this specification and describe the present invention in detail.

“Donepezil” stands for 1-benzyl-4-[(5,6-dimethoxy-1-indanon)-2-yl]methylpiperidine. The crystal of the salt or a solvate thereof according to the present invention may have crystal polymorphs. However, the crystal of the present invention should not be limited in terms of their crystal polymorphs, but may be a single crystal form or a mixture thereof.

Since errors may be generally caused in the range of ±0.2° in the diffraction angle (2θ) of powder X-ray diffraction, it is necessary that a value of the diffraction angle (2θ) mentioned above should be understood as including a numerical value in the range of around ±0.2°. Accordingly, not only the crystal having peak diffraction angles completely identical in powder X-ray diffraction but also crystals having peak diffraction angles identical in an error of around ±0.2° are included in the present invention.

“Alcohols” mean C₁₋₆ alkyl alcohols, and specific examples include methanol, ethanol, isopropanol, n-propanol, etc.

“Ethers” mean C₁₋₆ alkyl ethers or cyclic ethers, and specific examples include diethyl ether, diisopropyl ether, t-butyl methyl ether, tetrahydrofuran, etc.

In “1-benzyl-4-[(5,6-dimethoxy-1-indanon)-2-yl]methylpiperidine p-toluenesulfonate or a solvate thereof”, the solvate is not particularly limited as long as it is formed by the salt of the present invention and a solvent. It is a form of the compound in which the solvent forms salvation in an appropriate ratio between 0.1 and 5 molecules per one molecule of the compound. The solvent for solvate is not limited in particular, and example thereof includes a solvent used in the preparation of the salt of the present invention or the crystal thereof, or water, and preferably includes 1 to 3 solvents selected from the group consisting of water, diisopropyl ether and ethanol (a mixture at any arbitrary ratio in the case of a combination thereof) and more preferably includes water.

In the salt of the present invention, the ratio of donepezil to p-toluenesulfonic acid is not limited in particular, but p-toluenesulfonic acid forms a salt at a ratio of 0.5 to 2 molecules for one donepezil molecule (preferably about one molecule per one molecule of the compound).

The salt of the present invention or the solvate thereof and the crystal thereof can be prepared by the process described below. However, the process for preparing the salt of the present invention or the solvate thereof and the crystal thereof is not limited to these.

### Preparation Process

### (Preparation of Donepezil P-Toluenesulfonate)

In the process of the present invention, donepezil (1-benzyl-4-[(5,6-dimethoxy-1-indanon)-2-yl]methylpiperidine) to be used as a starting material can be prepared by a process described in Patent Document (WO99/29668A) and the like.

### (Operation 1)

Donepezil, a solvent and p-toluenesulfonic acid are mixed and dissolved at room temperature or by heating. In this dissolution step, the order of adding donepezil, the solvent and p-toluenesulfonic acid is not particularly limited and the operation can be performed with stirring or under still standing.

### (Operation 2)

After this mixed solution is dissolved, the salt of donepezil with p-toluenesulfonic acid can be obtained by the following process.

(1) Evaporating the solvent by placing the mixture under atmospheric pressure or under reduced pressure.
(2) Stirring or leaving the mixture at the dissolution temperature.
(3) Cooling the mixture from the dissolution temperature and stirring or leaving it.
(4) Adding an anti-solvent to the mixture at the dissolution temperature and agitating or leaving it.
(5) Adding an anti-solvent to the mixture at the dissolution temperature, cooling it and stirring or leaving it.

### (Operation 3)

When donepezil p-toluenesulfonate is obtained as a precipitate, solid substance and the like, the precipitate and the like can be washed with an appropriate solvent. The resulting precipitate or residue can be also dried as required at room temperature or by heating under atmospheric pressure or under reduced pressure.

The time of process to obtain donepezil p-toluenesulfonate (the above operation 2) is not limited in particular but preferably it is 1 hour to 3 days, and more preferably it is 1 to 24 hours. The cooling temperature and cooling rate to obtain donepezil p-toluenesulfonate are not limited in particular.

### (Preparation of Crystal of Donepezil P-toluenesulfonate)

Donepezil p-toluenesulfonate can be obtained as a crystal by performing the above (Operation 1) to (Operation 3).

Donepezil p-toluenesulfonate can be also obtained as a crystal by mixing and dissolving donepezil p-toluenesulfonate and a solvent, and then (1) evaporating the solvent by placing the mixture under atmospheric pressure or under reduced pressure; (2) stirring or leaving the mixture at the dissolution temperature; (3) cooling the mixture from the dissolution temperature and stirring or leaving it; (4) adding an anti-solvent to the mixture at the dissolution temperature and stirring or leaving it; (5) adding an anti-solvent to the mixture at the dissolution temperature, cooling it and stirring or leaving it.

p-Toluenesulfonic acid may be either solid or a solution but preferably it is p-toluenesulfonic acid monohydrate or p-toluenesulfonic acid.

The solvent mentioned above is not limited in particular but specific examples thereof include, for example, one or plural solvents selected from the group consisting of water, alcohols (for example, methanol, ethanol, isopropyl alcohol, etc.), esters (for example, methyl acetate, ethyl acetate, etc.), ketones (for example, acetone, etc.), nitriles (for example, acetonitrile, etc.), benzene, toluene, cyclic ethers (for example, dioxane, tetrahydrofuran, etc.), N,N-dimethylformamide, dimethyl sulfoxide, halocarbons (for example, methylene chloride, etc.). When plural solvents are used, they may be added as a mixed solvent or each solvent may be added separately.

The heating temperature when a mixture of donepezil and p-toluenesulfonic acid is dissolved in a solvent is not limited in particular but preferably it is 20 to 80° C.

The temperature at the time of cooling after dissolving a mixture of donepezil and p-toluenesulfonic acid in a solvent is not limited in particular but preferably it is −20 to 40° C.

The amount of the solvent used is not limited in particular but preferably it can be suitably selected so that the lower limit is the amount in which donepezil dissolves by heating and the upper limit is the amount which does not remarkably lower the yield of the crystal, and more preferably the volume ratio is 4 to 30 times to the weight of donepezil (v/w).

The amount of p-toluenesulfonic acid used is not limited in particular, as long as it is equal to or more than the equivalence of donepezil but preferably it is 1 to 3 times that of donepezil by molar ratio, and more preferably it is about 1 to 1.5 times that of donepezil by molar ratio.

When donepezil p-toluenesulfonate is to be obtained as a crystal, a seed crystal (a crystal of donepezil p-toluenesulfonate to be obtained) may be added before the precipitation of the crystal. The temperature at which the seed crystal is to be added is not specified in particular but preferably it is equal to or less than 60° C., and more preferably it is 10° C. to 40° C.

Before or during this process where the crystal precipitates, an anti-solvent (diethyl ether, isopropyl ether, t-butyl methyl ether, hexane, heptane, octane, a mixed solvent thereof, etc.) can be added appropriately.

Donepezil p-toluenesulfonate to be aimed at can be obtained by filtering the crystal precipitated in the liquid mixture.

The obtained crystal can be washed with the same solvent as the solvent used for dissolution if necessary. The resulting crystal can be dried at room temperature or by heating under atmospheric pressure or under reduced pressure if necessary.

For example, donepezil p-toluenesulfonate can be also obtained as a crystal by (1) adding a solvent (for example, water, alcohols (for example, methanol, ethanol, isopropyl alcohol, etc.), esters (for example, methyl acetate, ethyl acetate, etc.), ketones (for example, acetone, etc.), nitrites (for example, acetonitrile, etc.), benzene, toluene, cyclic ethers (for example, dioxane, tetrahydrofuran, etc.), N,N-dimethylformamide, dimethyl sulfoxide, halocarbons (for example, methylene chloride, etc.) or a mixed solvent thereof to the salt of donepezil with p-toluenesulfonic acid and heating and dissolving it at 20 to 80° C.; (2) adding diethyl ether, isopropyl ether, t-butyl methyl ether, hexane, heptane, octane, etc. to the mixture; and (3) cooling the mixture to −20 to 40° C. and separating by filtering the obtained precipitate.

The donepezil p-toluenesulfonate of the present invention or a solvate thereof or a crystal thereof is effective for the treatment, prevention, remission, improvement of various types of senile dementia; in particular, Alzheimer's type senile dementia, cerebrovascular disorders associated with cerebral apoplexy (cerebral hemorrhage, cerebral infarction), cerebral arteriosclerosis, head injury, etc.; aprosexia, disturbance of speech, hypobulia, attention deficit hyperactivity disorders, emotional disorders, memory disturbance, hallucinatory-paranoid states, behavioral changes, etc. associated with encephalitis, cerebral palsy, and the like.

Furthermore, the donepezil p-toluenesulfonate of the present invention or a solvate thereof or a crystal thereof has a potent and highly selective anticholinesterase effect and is useful as a drug based on these effects. Particularly, the donepezil p-toluenesulfonate of the present invention or a solvate thereof or a crystal thereof is useful for, for example, Huntington chorea, Pick's disease, late-onset aberration symptom in addition to Alzheimer's type senile dementia.

When the donepezil p-toluenesulfonate of the present invention or a solvate thereof or a crystal thereof is used as a drug, the said salt of the present invention or a solvate thereof or a crystal thereof and a suitable additive are generally mixed into a formulation, which is used. However, this does not exclude a possibility that the said salt of the present invention or a solvate thereof or a crystal thereof is used as a drug as it is.

The additive as mentioned above includes an excipient, binder, lubricant, disintegrant, colorant, flavoring agent, emulsifier, surfactant, solubilizer, suspending agent, isotonizing agent, buffer, preservative, antioxidant, stabilizer, sorbefacient or the like generally used for a drug and these can be appropriately combined together as desired.

When the donepezil p-toluenesulfonate of the present invention or a solvate thereof or a crystal thereof is used as a drug, it may be administered orally or parenterally. The dose varies depending on symptom severity; the age, sex, body weight, sensitivity of a patient; administration method; timing and interval of administration, properties, formulation and type of the pharmaceutical preparation; kind of the active ingredient and it is not limited in particular but preferably it is about 0.1 to 300 mg per adult per day, preferably about 1 to 100 mg per adult per day, which is usually administered once a day or dividedly 2 to 4 times a day.

In order to formulate as a preparation the donepezil p-toluenesulfonate of the present invention or a solvate thereof or a crystal thereof, it is formulated into a dosage form such as an injection, suppository, sublingual tablet, tablet, capsule, or percutaneous agent by a conventional method in the field of drug formulation. When an injection is prepared, a pH modifier, buffer, suspending agent, solubilizer, stabilizer, isotonizing agent, preservative and/or the like may be added to the principal agent as required and prepared into an intravenous, subcutaneous or intramuscular injection by a conventional method. On that occasion, they can be made into a lyophilizate by a conventional method if necessary.

Examples of the suspending agent include, for example, methyl cellulose, polysorbate 80, hydroxyethyl cellulose, gum Arabic, powdered tragacanth, carboxymethylcellulose sodium, and polyoxyethylene sorbitan monolaurate.

Examples of the solubilizer include, for example, polyoxyethylene hydrogenated castor oil, polysorbate 80, niacinamide, polyoxyethylene sorbitan monolaurate, magrogol, and castor oil fatty acid ethyl ester.

Examples of the stabilizer include, for example, sodium sulfite, sodium metasulfite, ether, and the like. Examples of preservative include, for example, methyl parahydroxybenzoate, ethyl parahydroxybenzoate, sorbic acid, phenol, cresol, chlorocresol, and the like.

Donepezil p-toluenesulfonate of the present invention or a solvate thereof or a crystal thereof can be produced, for example, by the method as described in the following Examples, and the effect of the compound can be confirmed by the method as described in a publication (JP-A-64-79151), etc. But these are illustrative, and the present invention is by no means limited to the following specific examples and it may be modified as long as it does not depart from the range of the present invention.

The term “room temperature” in the following Reference Examples and Examples usually refers to a temperature from about 10° C. to about 35° C. “%” refers to percent by weight unless otherwise specified.

### Example 1

240 mL of ethanol was added to 30 g of donepezil and then 18 g of p-toluenesulfonic acid monohydrate was added and warmed (external temperature 50° C.) to be dissolved. 450 mL of diisopropyl ether was added dropwise to the mixture with stirring at the internal temperature of 40° C. for 11 minutes (inner temperature when completed: 27° C.). The mixture was further stirred at room temperature for 15 hours and the precipitate was separated by filtration. After this precipitate was washed with 75 mL a of a mixed solution of ethanol-diisopropyl ether (8:15), it was vacuum dried at 50° C. for five hours and 42.3 g (yield 97.0%) of donepezil p-toluenesulfonate crystal was obtained.

### Measurement of Powder X-Ray Diffraction Pattern

The measurement of powder X-ray diffraction of the crystal obtained in each Example followed the powder X-ray diffractometry described in the general test method of Japanese Pharmacopeia and was performed under the following measurement conditions.

### (Device)

Rigaku X-ray DTA system: RINT-2000 (made by Rigaku Corporation)

### (Operation Method)

Measurement of samples was performed under the following conditions.

X-ray used: CuKα ray
Tube voltage: 40 kV
Tube current: 200 mA
Divergence slit: ½ deg
Receiving slit: 0.3 mm
Scattering slit: ½ deg
Scanning speed: 2°/min
Scanning step: 0.02°
Measurement range (20): 5 to 40°

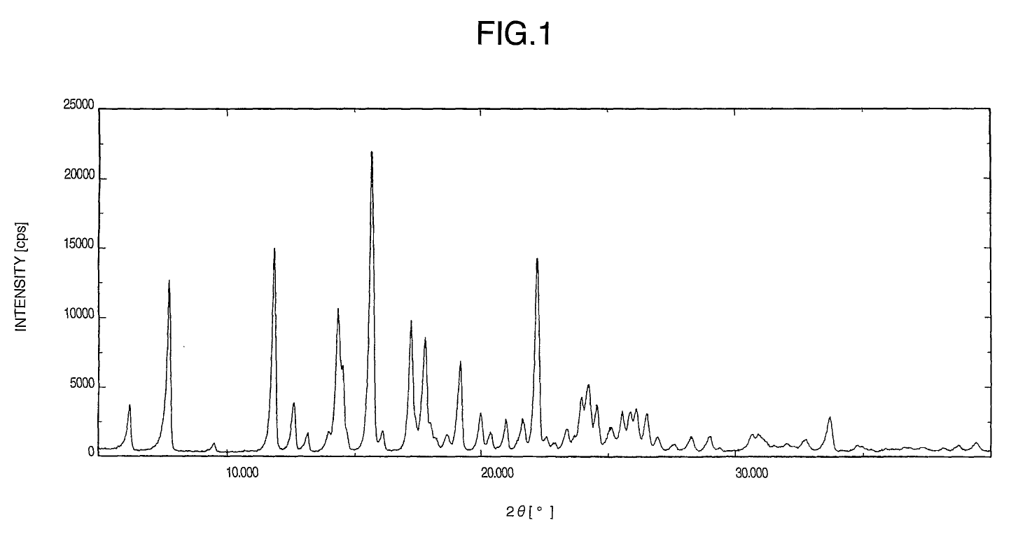

The powder X-ray diffraction pattern of the crystal obtained in Example 1 is shown in FIG. 1.

### INDUSTRIAL APPLICABILITY

Donepezol p-toluenesulfonate or a solvate thereof, or a crystal thereof of the present invention has excellent acetylcholine esterase inhibitory effect and therefore, it is useful as a drug, particularly a preventive or therapeutic agent for various types of senile dementia, etc. taking advantage of the acetylcholinesterase inhibitory effect.

1. 1-Benzyl-4-[(5,6-dimethoxy-1-indanon)-2-yl]methylpiperidine p-toluenesulfonate or a solvate thereof.

2. A crystal of 1-benzyl-4-[(5,6-dimethoxy-1-indanon)-2-yl]methylpiperidine p-toluenesulfonate or a solvate thereof having diffraction peaks at diffraction angles (2θ=0.2°) of 15.7° and 22.2° in powder X-ray diffraction.

3. The crystal according to claim 2, wherein the crystal has further diffraction peaks at diffraction angles (2θ±0.2°) of 7.8° and 14.3° in powder X-ray diffraction.

4. A process for preparing the crystal according to claim 2, characterized in that crystallization is performed using one or two solvents selected from the group consisting of an alcohol solvent, an ether solvent and water.
